(19) United States
(12) Patent Application Publication
(10) Pub. No.: US 2005/0100880 A1
(43) Pub. Date: May 12, 2005

(54) Biosensor test strips of multiple function for multiple uses

(76) Inventor: Yu-Hong Chang, San-Chung City (TW)

Correspondence Address: Yu-Hong Chang, P.O. Box No. 6-57, Junghe, Taipei, 235, TW

(21) Appl. No.: 10/704,701

(22) Filed: Nov. 12, 2003

(51) Int. Cl. C12Q 1/00; G01N 27/26

(52) U.S. Cl. 435/4; 204/403.03

(57) ABSTRACT

This invention discloses a detachable test strip good for multiple tests on one strip to reduce the cost of test strip per test, and a test strip with more than two electrodes for the simultaneous measurement of more than one biological data with one drop of blood only.

### BACKGROUND OF THE INVENTION

a) Field of the Invention

The present invention generally relates to a new biosensor test strip and, more particularly, to the measurement of blood glucose, uric acid and cholesterol, etc., which will be cost and functional competitive against the current suppliers.

b) Discussion of the Prior Art

All the currently available biosensor test strips, whether they are for the measurement of blood glucose, uric acid, or cholesterol, use one strip to perform one test, which are often very expensive for the end users. There are a lot of patients, especially in the developing countries, who are too poor to pay for the DIY tests at home, are in urgent need for a cheaper test strip. Moreover, all current test strips can only give one test result from one test strip, to have different readings of, say, blood glucose, uric acid and cholesterol, requires several individual tests and several blood inoculation which are very painful for the patients. It is therefore the objective of the present invention to come up with a new design of test strip, which can perform more than one test results out of one test strip from one blood inoculation only. It is also another objective of the present invention to design a new test strip with much lower cost for the end users.

### SUMMARY OF THE INVENTION

In view of the need for a much cheaper test strip and the need for a test strip capable of performing more than one item of tests with one drop of blood, we therefore design a new detachable test strip, which has multiple test reagents embedded on a single strip. After a good test, the tested section of the strip can be broken off and the other fresh un-tested area with reagents can be kept for further re-use. We also re-design the layout of the conductive electrodes to allow more than one reagents to be embedded on the same designated spot of the test strip for the inoculated blood. Thus, with the application of one drop of blood, several test results can be measured from the data acquired from the specially designed layout of electrodes.

### BRIEF DESCRIPTION OF THE PREFERRED EMBODIMENT

The disposable and detachable test strips of the present invention may be more readily understood by one skilled in the art with reference to the following detailed description taken in conjunction with the accompanying drawings wherein like elements are designated by identical reference numbers throughout the several views and in which:

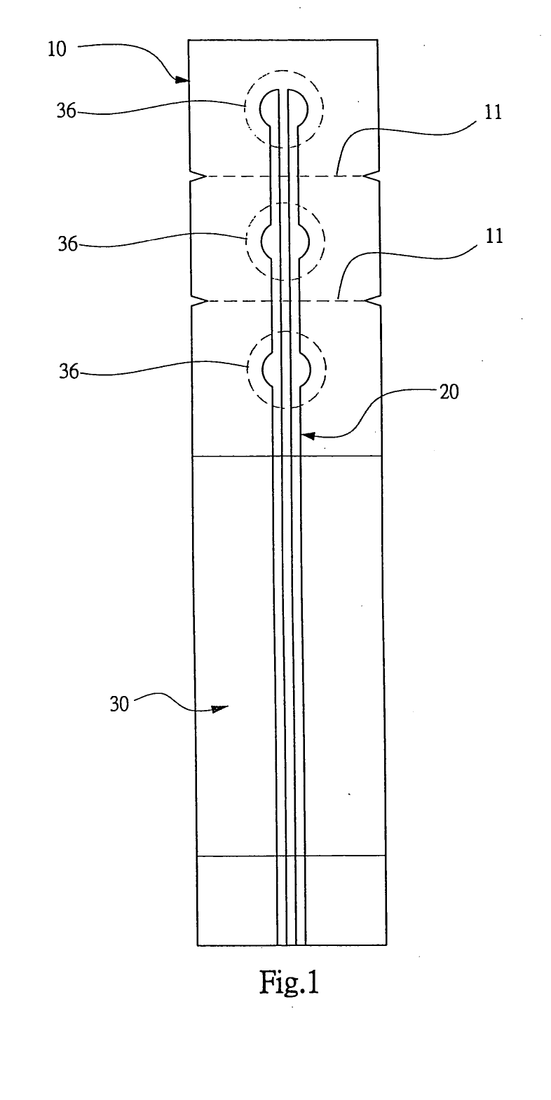

FIG. 1 is the schematic view of the detachable test strip with two conductive electrodes, good for three tests, but one reading per test only.

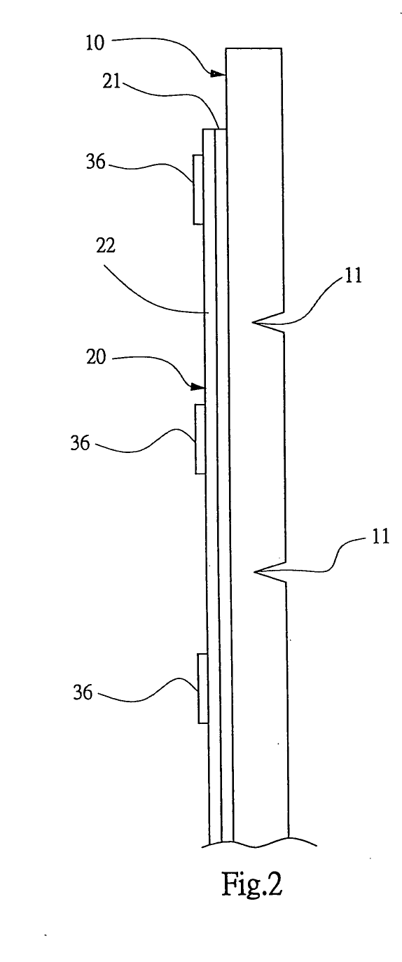

FIG. 2 is the side view of the detachable test strip showing the composition of the conductive electrodes, which includes a layer of conductive silver and a layer of conductive carbon and on which lies an reagent.

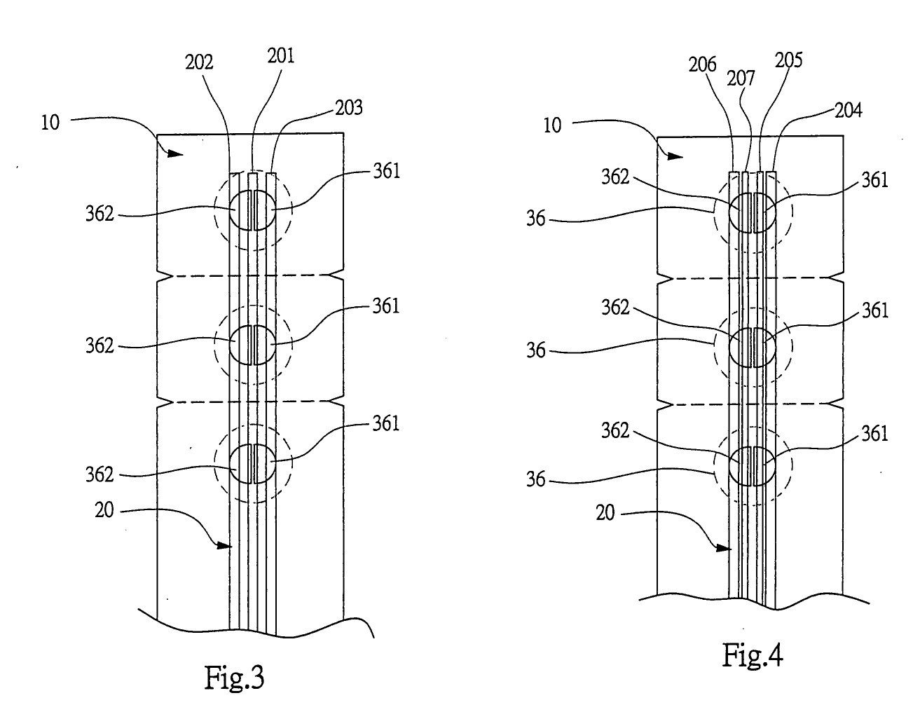

FIG. 3 illustrates the schematic view of the detachable test strip with three conductive electrodes, good for three tests, but two test readings per test.

FIG. 4 illustrates the schematic view of the detachable test strips with four conductive electrodes, good for three tests per strip, but two different test readings per test.

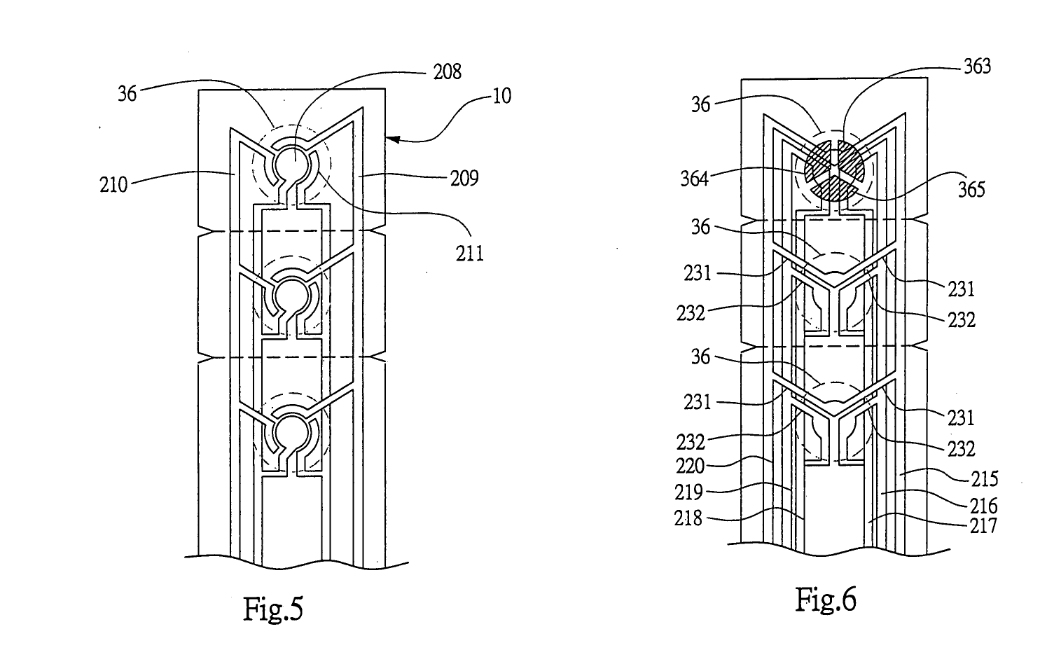

FIG. 5 gives the schematic view of the detachable test strips with four conductive electrodes, good for three tests per strip, but three different test readings per test.

FIG. 6 gives the schematic view of the detachable test strips with six conductive electrodes, good for three tests per strip, but three different test readings per test.

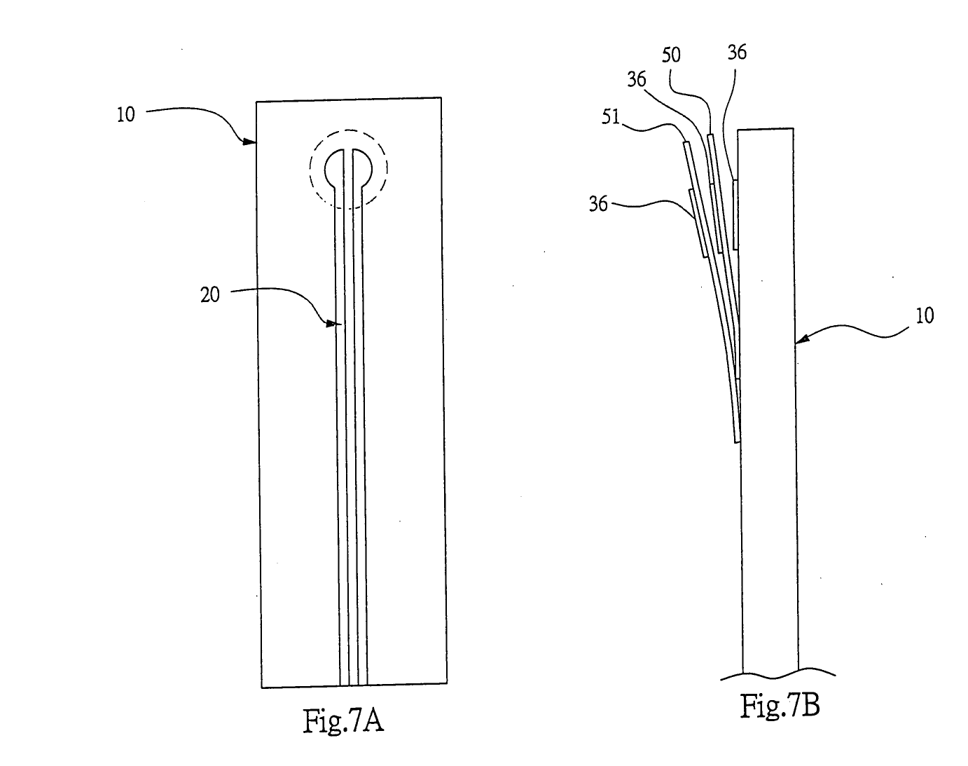

FIG. 7A & 7B indicates another type of detachable test strips for multiple tests, in addition to the detachable device of FIG. 6.

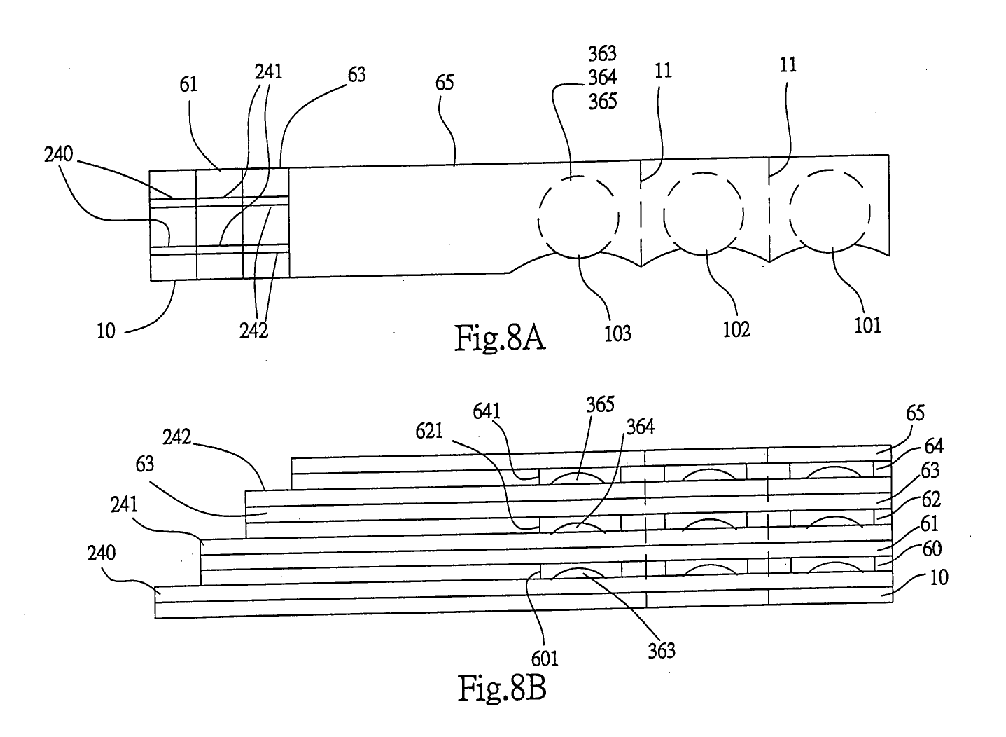

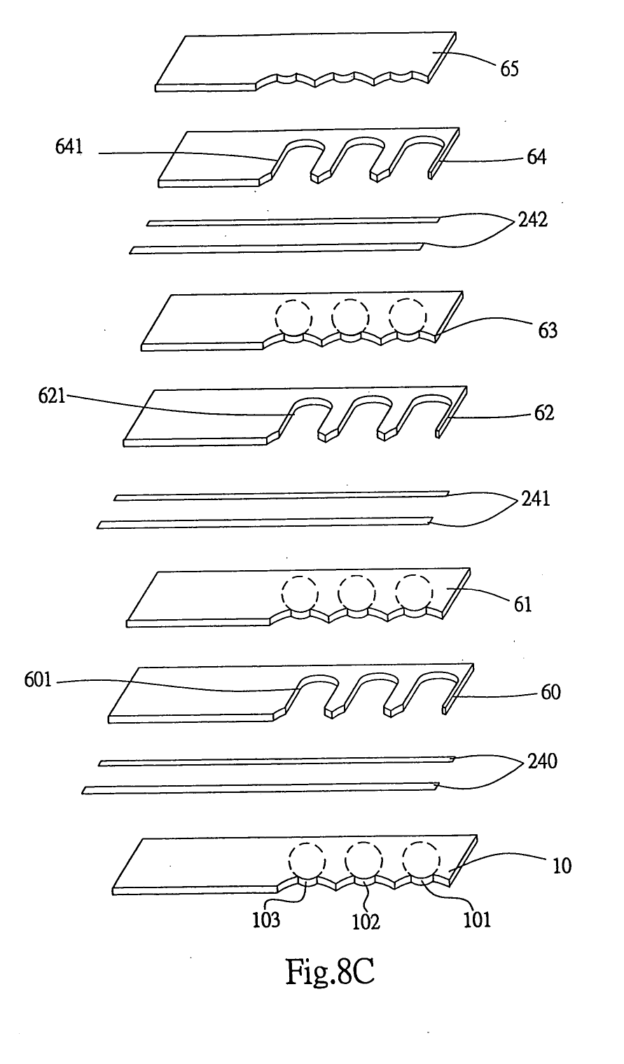

FIGS. 8A, 8B and 8C further indicates another type of detachable test strips for multiple tests in addition to the devices of FIG. 6 and FIG. 7.

### DETAILED DESCRIPTION OF THE PREFERRED EMBODIMENT

Referring to the drawing in detail, FIG. 1 is a schematic view of the detachable test strips with two conductive electrodes, good for performing three tests, but there will be one reading per test only. As a drop of blood is added to the dashed circular area for reagents 36 of the strip 10, the pre-embedded reagent 36 reacts electrochemically with the blood to produce an electric current, which will be measured through the conductive electrodes to determine the biological readings of interest in the blood. After a test is done with the test, the tested section can be easily broken off from the dashed line 11 which has a pre-cut across the width of the test strip 10 on the opposite side of the test strip 10, as shown in FIG. 2. FIG. 2 also shows the composition of the electrodes as one layer of conductive silver 21, on top of which lies a layer of conductive carbon. Reagents 36 are placed on top of the carbon electrode. FIG. 3 shows the schematic view of the drawings of the detachable test strips with three electrodes, good for performing three tests, but there will be two test readings per test. Reagent 361 is different from reagent 362 to produce two different biological test data with one drop of blood which could cover very well both regents 361 and 362. The central electrode 201 serves as the ground reference electrode for the neighboring electrodes 202 and 203. FIG. 4 further discloses the schematic view of the detachable test with four electrodes, good for three tests per strip but two test reading per test. Electrodes 204 and 205 are used to measure one reaction, just like electrodes 206 and 207. In order to perform three different measurement on one test piece, there are two different arrangement of electrodes, as shown in FIG. 5 with four electrodes, and in FIG. 6 with six electrodes. FIG. 5 uses the electrode 208 as the ground reference electrodes for electrodes 209, 210 and 211. FIG. 6 illustrates the schematic view of the detachable test strip with six electrodes, wherein the clechodes 215 and 216 are used to measure the electrochemical reaction of the reagent 363, while the electrodes 216 and 218 are used to measure the electrochemical reaction of the reagent 365, and the electrodes 219 and 220 are used to measure the reagent 364. In FIG. 6, although the branch electrode 231 of the electrode 215 crosses over the electrode 216, it separates from the electrode 217 at the intersection with an electric insulation layer, so is the branch electrode 232 with the electrode 217 and so are the electrodes 231 and 232 with the electrodes 219 an 218 respectively. The area 30 is covered with and insulation material against contamination with foreign objects.

FIGS. 7A & 7B further discloses another type of multiple detachable test strips. This is done by attaching a few thin films 50 and 51 of test strips on the base strip 10. Both thin film of strips, 50 and 51, and the strip 10 all have conductive electrodes and reagents 36.

FIGS. 8A, 8B and 8C further give the top, side and exploded views of another type of detachable test strips for multiple tests. Reagents 363, 364 and 365 are placed on the test strips 10, 61 and 63, on top of which are coated with two conductive electrodes 240, 241 and 242, respectively, to pick up the electric current from the electrochemical reaction of the reagents with the applying blood. The strips 64, 62 and 60 all have three horseshoe cuts 641, 621 and 601 to accommodate the reagents for electrochemical reaction, while the strips 61 and 63 serve as the insulation layer for the electrodes 240, 241 and 242, and the strip 65 serves as the protective layer to accommodate the reagents 363, 364 and 365 within the horseshoe cuts 641. The undercuts 101, 102 and 103 are the place to apply one blood drop from the side for reaction with three different reagents. The electrodes 240, 241 and 242 are insulated from each other by the strips 60, 61, 62, 63, 64 and 65. This whole test strip 10 is designed to the fresh, non-reacted portion.

1. A test strip with several detachable test pieces, comprising of:
a) a test strip with pre-cut under the base strip ready to be broken off after use, with conductive electrodes and reagents laid on the surface thereof;
b) a test strip with a plurality of thin film of test piece on the base strip, attached with conductive electrodes and reagents. After a layer of thin film is done with test, it will be peeled off for another fresh thin film with reagents and electrodes.

2. A test strip with more than two conductive electrodes for simultaneous measurement of multiple biological tests, comprising of:
a) three electrodes for the measurement of two different reagents, with the central electrodes serving as the ground reference electrodes;
b) four electrodes for measuring two different reagents, with two electrode for one regent;
c) four electrodes for measuring three different reagents, with one electrode serving as ground reference electrode;
d) six electrodes for measuring three different reagents, with no electrode serving as ground reference electrode;
e) more than six electrodes for the measurement of more than three reagents.

3. A test strip with multi-layered electrodes for the simultaneous measurement of several biological tests, comprising of:
a) one or more than one layer of two conductive electrodes with base strip, on which are embedded with one or more than one reagents, stacked together with glue or firmly bonded by ultrasonic device;
b) a test strip with one or more than one pre-cuts ready to be broken off or cut off after every use.
